(19) United States
(12) Patent Application Publication
(10) Pub. No.: US 2025/0169866 A1
(43) Pub. Date: May 29, 2025

(54) IMPLANT DRIVER SYSTEMS AND RELATED SURGICIAL METHODS

(71) Applicant: Alex HERZLINGER, Chadds Ford, PA (US)

(72) Inventor: Alex HERZLINGER, Chadds Ford, PA (US)

(21) Appl. No.: 18/957,077

(22) Filed: Nov. 22, 2024

Related U.S. Application Data
(60) Provisional application No. 63/602,558, filed on Nov. 24, 2023.

(51) Int. Cl. A61B 17/88; A61B 90/00

(52) U.S. Cl. CPC A61B 17/8875; A61B 2090/031

(57) ABSTRACT

The disclosure an implant driver designed for bone fixation during surgical procedures. The implant driver includes a body with a handle, an attachment member, and an external clip for holding multiple implants. A driver mechanism is integrated into the body to sequentially feed and implant the plurality of implants into the surgical site. The driver is activated by a control element, which can be a button or trigger. The external clip is enclosed in a sterilizable casing to prevent contamination, and the system may include various safety and adjustment features for optimal performance.

### CROSS-REFERENCE TO RELATED APPLICATIONS

The present application claims the benefit of and priority to U.S. Provisional Patent Application No. 63/602,558, filed Nov. 24, 2023, the entire disclosure of which is incorporated by references into the present application.

### FIELD OF THE INVENTION

The present disclosure relates to surgical instruments and, more specifically, to an implant driver for bone fixation and related surgical methods.

### BACKGROUND

The fixation of bone segments during surgical procedures often involves the use of implants such as screws. Efficient and accurate implantation is crucial for successful bone fixation. Currently, bone screws are inserted and driven into surgical site one screw at time using a driver. The screw is held in position and the driver drives the screw in place. The surgeon then grabs another screw and the process repeats until fixation is complete. In some instances, especially for cranial fixation, the screws are quite small and managing multiple screws one at time can be cumbersome.

### SUMMARY

The present disclosure includes embodiments of a bone fixation system that includes an implant driver and implants that enhances the precision and ease of the bone fixation process. An embodiment of the present disclosure includes an implant driver for bone fixation at a surgical site. The implant driver includes a body having a handle. The implant driver further includes an attachment member on the body, the attachment member configured to removably receive and secure an external clip. The implant driver further includes an external clip configured to hold a plurality of implants, the external clip removably attached to the attachment member, the external clip configured to feed one or more implants in sequence into the body for implantation into the surgical site. The implant driver further includes a driver carried by the body and coupled to the external clip, the driver configured to engage and drive one or more of the plurality of implants from the external clip toward the surgical site. The implant driver further includes a sterilizable casing that encloses the external clip. The implant driver further includes a control element configured to activate the driver to drive the one or more of the plurality of implants into the surgical site.

A further embodiment of the present disclosure includes an implant driver. The implant driver includes a body having a handle that extends perpendicular to the body. The implant driver further includes a barrel assembly mounted to the body. The implant driver further includes a clip located in the handle, the clip comprising a magazine for holding a plurality of implants, the clip configured to feed the implant into the barrel for implantation. The implant driver further includes a driver operatively connected to the clip, the driver configured to engage and eject the plurality of implants from the clip into a surgical site. The implant driver further includes a sterilizable casing configured to enclose the clip. The implant driver further includes a control element carried by the handle and configured to activate movement of the driver to drive the implant into the surgical site.

A further embodiment of the present disclosure includes an implant driver. The implant driver includes a body having a proximal end and a distal end opposite the proximal end, the body including a grip disposed at the proximal end that extends perpendicular to the body. The implant driver further includes a barrel assembly mounted to the body. The implant driver further includes a circular clip disposed at the distal end of the body, the clip configured to hold a plurality of implants, the clip configured to feed the implant into the barrel for implantation. The implant driver further includes a driver operatively connected to the clip, the driver configured to engage and eject the plurality of implants from the clip into a surgical site. The implant driver further includes a sterilizable casing configured to enclose the clip. The implant driver further includes a control element carried by the handle and configured to activate movement of the driver to drive the implant into the surgical site.

A further embodiment of the present disclosure includes an implant driver. The implant driver includes a body having a proximal end and a distal end opposite the proximal end, the body including a grip disposed at the proximal end that extends perpendicular to the body. The implant driver further includes a barrel assembly mounted to the body. The implant driver further includes a plurality of circular clips disposed at the distal end of the body, the plurality of clips configured to hold a plurality of implants, the clips configured to feed the implant into the barrel for implantation. The implant driver further includes a driver operatively connected to the plurality of clips, the driver configured to engage and eject the plurality of implants from the plurality of clips into a surgical site. The implant driver further includes a sterilizable casing configured to enclose the plurality of clips. The implant driver further includes a control element carried by the handle and configured to activate movement of the driver to drive the implant into the surgical site.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown:

FIG. 1 is a prospective view of an implant driver according to an embodiment of the present disclosure;

FIG. 2 is a side view of an implant driver according to an embodiment of the present disclosure;

FIG. 3 is a side view of an implant driver according to an embodiment of the present disclosure;

FIG. 4 is a prospective view of an implant driver according to an embodiment of the present disclosure; and

FIG. 5 is a side view of an implant driver according to an embodiment of the present disclosure.

### DETAILED DESCRIPTION OF ILLUSTRATIVE EMBODIMENTS

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, left, right, above, below and diagonal, are used with respect to the accompanying drawings. Such directional terms used in conjunction with the following description of the drawings should not be construed to limit the scope of the present disclosure in any manner not explicitly set forth.

Embodiments of the present disclosure include implant drivers and related systems and methods for bone fixation. The implant drivers as described herein may include external clips or cartridges that hold multiple implants, such as bone screws, for use in bone fixation whereby two or more bone segments are coupled together. Typically bone fixation uses bone screws in combination with bone plates. Here, the implant drivers are configured to present the bone screw to the target site and to also drive the bone screw into the target site, whether that is bone or a bore or through-hole in a bone plate. Thus, the implant driver as described herein may form part of bone fixation system that also include different bone plates configured for a particular indication. The implant drivers may deliver several bone screws in sequence without the need to reload the driver. In addition, the implant driver can also be configured as so-called autoloading devices. The implant drivers as described herein are suited for cranial fixation in particular. However, the implant drivers can be used in other areas of bone fixation, such as long bone fixation, extremities, etc.

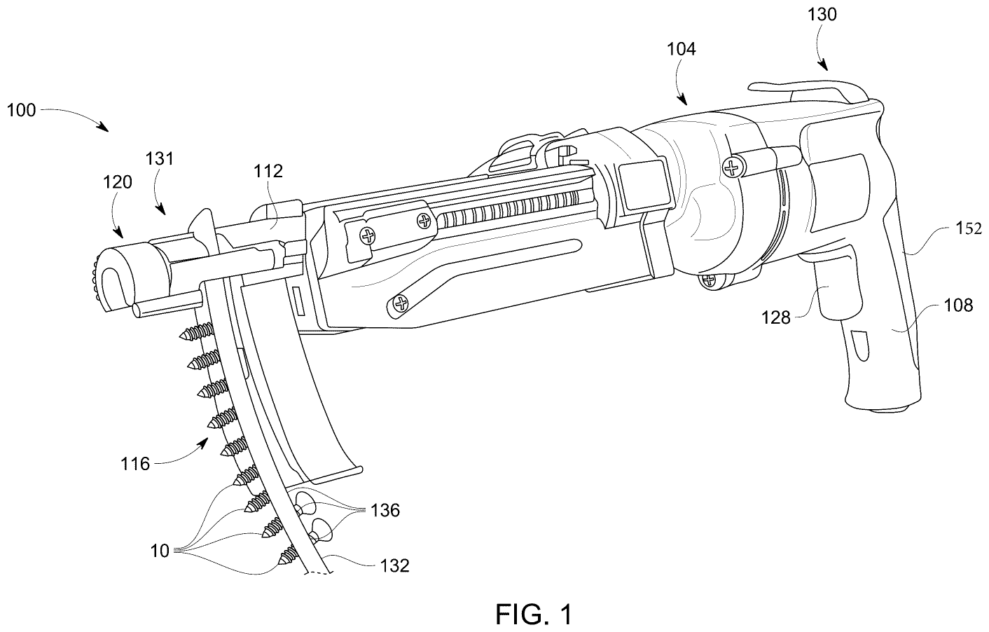

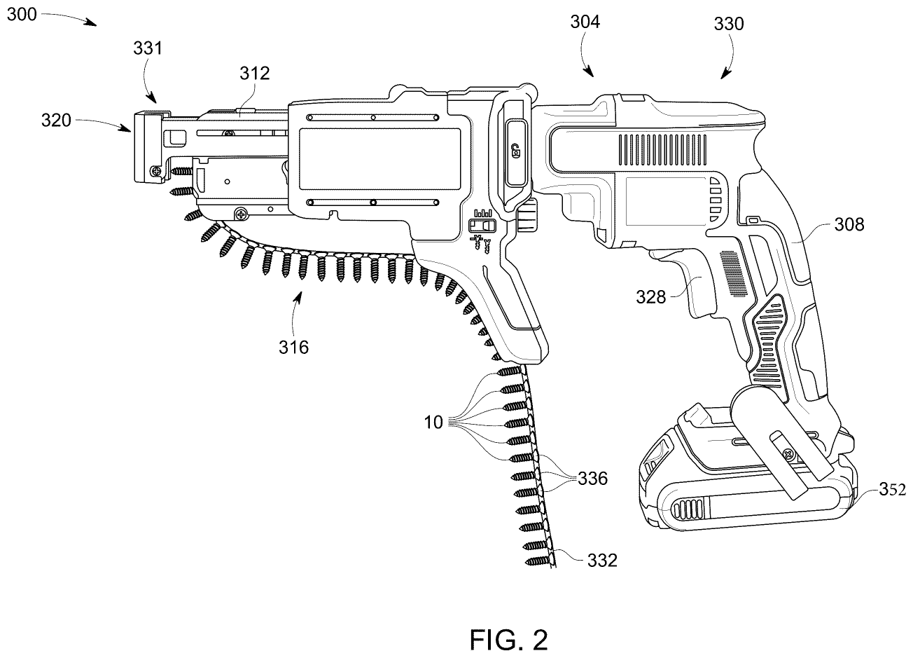

Referring to FIGS. 1 and 2, an implant driver 100, 300 is configured for bone fixation at a surgical site. The implant driver 100, 300 includes a body 104, 304 having a grip or handle 108, 308, an attachment member 112, 312 on the body 104, 304, an external clip 116, 316, a driver 120, 320 (not depicted), a sterilizable casing 124, 324, and a control element 128, 328. The body 104, 304 also includes a proximal end 130, 330 that carries the handle 108, 308 and a distal end 131, 331 opposite the proximal end 130, 330, the distal end 131, 331 including the attachment member 112, 312 and the driver 120, 320. In one embodiment, the distal end 131, 331 receives the attachment member 112, 312 and the driver 120, 320. The attachment member 112, 312 is configured to removably receive and secure the external clip 116, 316 to the implant driver 120, 320.

As shown in FIGS. 1 and 2, the external clip 116, 316 is configured to hold a plurality of implants 10. The external clip 116, 316 is removably attached to the attachment member 112, 312 and may be arranged generally perpendicular to the body 104, 304. The attachment member 112, 312 is configured to feed one or more implants 10 in sequence into the body 104, 304 for implantation into the surgical site. Here, the external clip 116, 316 arranges the implants 10 in a side-by-side relationship. The external clip 116, 316 may include a clip body 132, 332 with a plurality of engagement members 136, 336 arranged in series along a length of the clip body 132, 332. The plurality of engagement members 136, 336 are configured to removably secure the implants 10 in place on the clip 116, 316, such that the head of the implant 10 is located along one side the clip body 132, 332 and the shaft extends through the clip body 132, 332. As the clip 116, 316 advances, the shaft and head are aligned with the driver 120, 320 for driving into the surgical site. The external clip 116, 316 can hold between five and twenty implants, depending on the implant size and need. However, more than twenty implants could be used. FIG. 2 show an alternative embodiment of an external clip.

The driver 120, 320 is carried by the body 104, 304 and coupled to the external clip 116, 316. The driver 120, 320 is configured to engage and drive one or more of the plurality of implants 10 from the external clip 116, 316 toward the surgical site. In the illustrated embodiment, the driver 120, 320 is configured to rotate once the driver tip 140, 340 engages the head of the shaft of the implant 10. Once rotation begins, torque applied to the implant forces the implant 10 further into the surgical site. The external clip 116, 316, while advancing the implants 10 in sequence, can aid in presenting the implants 10 for positioning at the surgical site, at which point the driver 120, 320 engages the head and rotates, causing the implant 10 to rotate.

The sterilizable casing 124, 324 encloses the external clip 116, 316. The sterilizable casing 124, 324 is configured to inhibit contamination of the external clip 116, 316 whether the external clip 116, 316 is attached to the attachment member 112, 312 or not. In this example, the casing can be any material or barrier that maintains biomedical sterility while still providing access for the anchors to engage and exit the driver during use. This allows the cartridges to maintained a sterile environment but allow for the modular use of the product in use.

The control element 128, 328 is configured to activate the driver 120, 320 to drive the one or more of the plurality of implants 10 into the surgical site. The control element 128, 328 includes a clutch 144, 344 that disengages the driver 120, 320 when a predetermined torque level is reached. The control element 128, 328 is further configured to adjust the speed and direction of the driver 120, 320 in response to user input. The control element 128, 328 includes a safety interlock mechanism 148, 348 to prevent accidental activation of the driver 120, 320. In one embodiment, the control element 128, 328 is a button. In another embodiment, the control element 128, 328 is a trigger. However, any particular control mechanism could be used. In one embodiment, the control element 128, 328 includes a clutch mechanism that disengages the driver 120, 320 when a predetermined torque level is reached. The control element 128, 328 is further configured to adjust the speed and direction of the driver 120, 320 in response to user input. The control element 128, 328 further includes a battery 152, 352 configured to power the driver 120, 320.

The implant driver 100, 300 further includes a depth adjustment mechanism 156, 356 for controlling the depth at which the plurality of implants 10 are driven into the surgical site.

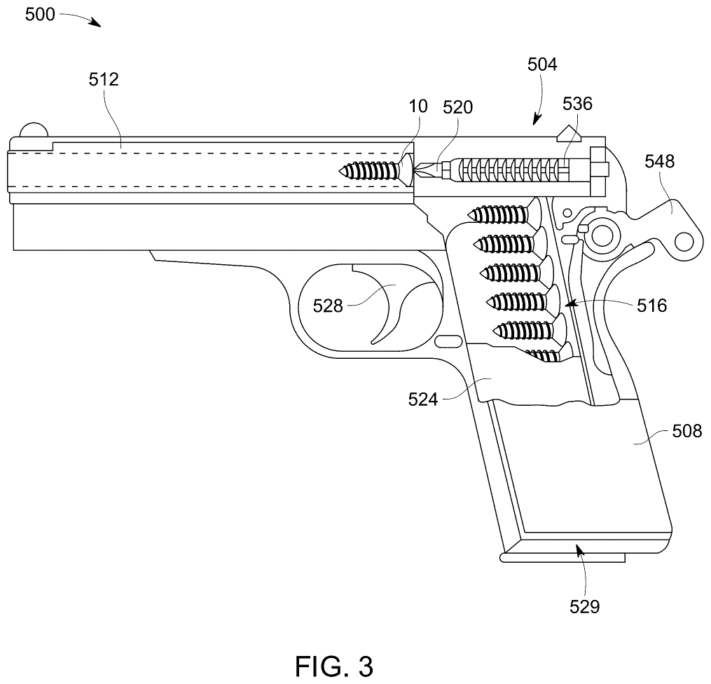

Referring to FIG. 3, an implant driver 500 includes a body 504 having a handle 508 that extends perpendicular to the body 504, a barrel assembly 512 mounted to the body 504, a clip 516, a driver 520, a sterilizable casing 524, and a control element 528.

The clip 516 is located in the handle 508. The clip 516 comprises a magazine 529 for holding a plurality of implants 10, the clip 516 is configured to feed the implant 10 into the barrel 512 for implantation. In the illustrated embodiment, the clip 516 is configured to hold at least six implants, such as between six and twelve implants. The clip 516 includes a spring loaded advancement device 536 inside the casing 524 that advances the implants 10 into the barrel assembly 512 as implants 10 are ejected and placed in the surgical site.

The driver 520 is operatively connected to the clip 516 and carried by the barrel assembly 512. The driver 520 is configured to engage and eject the plurality of implants 10 from the clip 516 into a surgical site. The sterilizable casing 524 encloses the clip 516 and is configured to sterilize the plurality of implants 10 when the clip 516 is loaded into the handle 508.

The control element 528 is carried by the handle 508 and configured to activate movement of the driver 520 to drive the implant 10 into the surgical site. In one embodiment, the control element 528 is a trigger. The control element includes a safety interlock mechanism 548 to prevent accidental activation of the driver 520. In the embodiment shown, the control element 528 is a trigger.

The implant driver 500 further includes a depth adjustment mechanism 556 and a recoil spring assembly 560. The depth adjustment mechanism 556 is configured to control the depth at which the plurality of implants 10 are driven into the surgical site. The recoil spring assembly 560 is configured to activate the driver 520 and eject the plurality of implants 10.

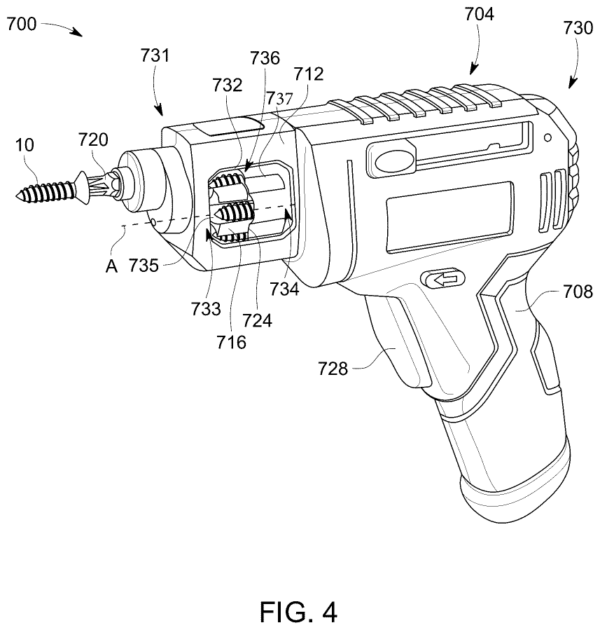

Referring to FIG. 4, an implant driver 700 includes a body 704, a barrel assembly 712, a circular clip 716, a driver 720, a sterilizable casing 724, and a control element 728. The body 704 has a proximal end 730 and a distal end 731 opposite the proximal end 730. The body 704 includes a handle or grip 708 disposed at the proximal end 730 that extends perpendicular to the body 704. The barrel assembly 712 is mounted to the body 704.

The circular clip is disposed at the distal end of the body 704. The clip 716 is configured to hold a plurality of implants 10 and feed the implant 10 into the barrel 712 for implantation. The clip 716 includes a clip body 732 having a forward end 733, a rear end 734, and a central axis A that extends through the forward and rear ends 733, 734. The clip body 732 includes a plurality of implant channels 735 and engagement members 736 that are disposed around the central axis A. The implant channels 735 each hold a respective implant 10 while the engagement members 736 are located in the implant channels 735 and hold the implants 10 in place. The clip 716 also includes a shaft 737 engaged with barrel assembly 712 that allows the clip 716 to rotate as the implants 10 are used. As the clip 716 rotates into position, a driver 720 can engage the implant 10 and advance it out of the implant channel 735 into the surgical site, while also rotating the implant 10. In the illustrated embodiment, the clip 716 is configured to hold at least six implants though more could be held by the clip 716 as needed.

The driver 720 is operatively connected to the clip 716. The driver 720 is configured to engage and eject the plurality of implants 10 from the clip 716 into a surgical site, as described above.

The sterilizable casing 724 is configured to enclose the clip 716. The sterilizable casing 724 is configured to sterilize the plurality of implants 10 when the clip 716 is attached to the distal end 731 of the body 704.

The implant driver 700 as shown in FIG. 4 also includes a depth adjustment mechanism 756 for controlling the depth at which the plurality of implants 10 are driven into the surgical site.

The control element 728 is carried by the grip 708 and configured to activate movement of the driver 720 to drive the implant 10 into the surgical site. In the illustrated embodiment, the control element 728 includes a button. The control element 728 includes a safety interlock mechanism 748 to prevent accidental activation of the driver 720.

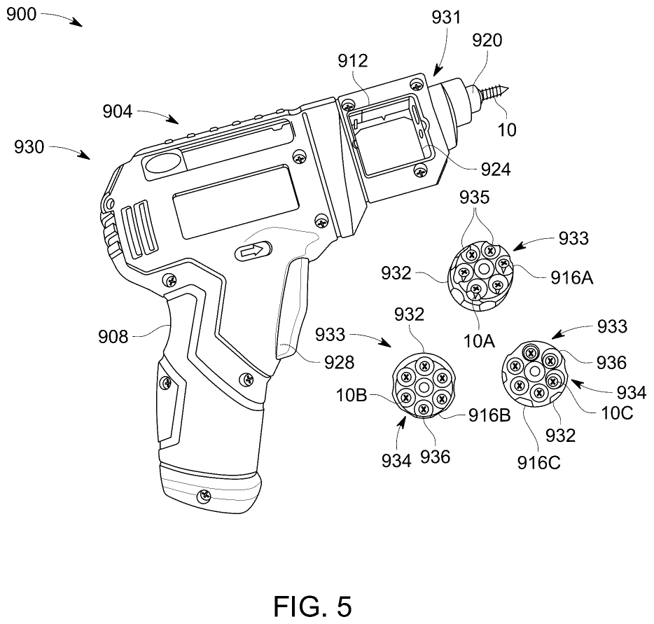

Referring to FIG. 5, an implant driver 900 includes a body 904 having a proximal end 930 and a distal end 931 opposite the proximal end 930, a barrel assembly 912 mounted to the body 904, a plurality of circular clips 916 configured to be disposed at the distal end 931 of the body 904, a driver 920, a sterilizable casing 924, and a control element 928. The body 904 includes a handle or grip 908 disposed at the proximal end 930 that extends perpendicular to the body 904.

The plurality of clips 916 is configured to hold a plurality of implants 10 and feed the implant 10 into the barrel 912 for implantation. In the illustrated embodiment, the plurality of clips 916 are preloaded with at least six implants. Furthermore, in the illustrated embodiment, the plurality of clips are circular clips. The plurality of clips 916 each includes a clip body 932 having a forward end 933, a rear end 934, and a central axis B that extends through the forward and rear ends 933, 934. The clip body 932 includes a plurality of implant channels 935 and engagement members 936 that are disposed around the central axis B. The implant channels 935 each hold a respective implant 10 while the engagement member 936 located in the implant channels 935 hold the implants 10 in place. The plurality of clips 916 also include a shaft 937 (not depicted) engaged with the barrel assembly 912 that allows the clips 916 to rotate as the implants 10 are used. As the clips 916 rotate into position, a driver 920 can engage the implant 10 and advance it out of the implant channel 935 into the surgical site, while also rotating the implant 10. In the illustrated embodiment, the plurality of clips 916 are configured to hold at least six implants though more could be held by the clips as needed.

The plurality of clips 916 are interchangeable in that a first clip 916A may have a first size implant 10A, a second clip 916B would have a second sized implant 10B and a third clip 916C would have a third sized implant 10C. Thus, one instrument can be used across multiple implants 10, e.g. bone screws.

The driver 920 is operatively connected to the plurality of clips 916. The driver 920 is configured to engage and eject the plurality of implants 10 from the plurality of clips into a surgical site.

The sterilizable casing 924 is configured to enclose the plurality of clips 916. The sterilizable casing 924 is further configured to sterilize the plurality of implants 10 when the plurality of clips 916 are attached to the distal end 931 of the body 904.

The control element 928 is carried by the grip 908 and configured to activate movement of the driver 920 to drive the implant 10 into the surgical site. In the illustrated embodiment, the control element 928 includes a button. The control element includes a safety interlock mechanism 948 to prevent accidental activation of the driver 920.

The implant driver 900 shown in FIG. 5 further includes a depth adjustment mechanism 956 for controlling the depth at which the plurality of implants 10 are driven in the surgical site.

In any of the embodiments disclosed, the plurality of implants 10 include bone screws. For example, the implants 10 are locking screws or compression screws. Accordingly, in any of the embodiments disclosed, the bone screws include a head, a shaft that extends from the head, an optional cannulation. In one embodiment, the shaft is completely threaded. In another embodiment, sections of the shaft between the distal tip and distal surface of the head are unthreaded and smooth. In other configurations, the outer surface of the head is threaded, and an entirety of the shaft is threaded. The implants 10 as described herein have a diameter that ranges from 1.5 mm to about 5.0 mm. The implants 10 have a length that ranges from 8.0 mm to about 18.0 mm.

Continuing with FIGS. 1-5, a method for fixing one or more bone segments together is disclosed. The method includes placing a fixation member in position at the surgical site, and inserting one or more implants 10 into the fixation member and the one or more bone segments using the implant driver 100, 300, 500, 700, 900 according to any of the embodiments disclosed.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

1. An implant driver for bone fixation at a surgical site, comprising:
a body having a handle;
an attachment member on the body, the attachment member configured to removably receive and secure an external clip;
an external clip configured to hold a plurality of implants, the external clip removably attached to the attachment member, the external clip configured to feed one or more implants in sequence into the body for implantation into the surgical site;
a driver carried by the body and coupled to the external clip, the driver configured to engage and drive one or more of the plurality of implants from the external clip toward the surgical site;
a sterilizable casing that encloses the external clip; and
a control element configured to activate the driver to drive the one or more of the plurality of implants into the surgical site.

2. The implant driver of claim 1, wherein the body further comprises a proximal end that carries the handle and a distal end opposite the proximal end, the distal end including an attachment member and the driver.

3. (canceled)

4. (canceled)

5. (canceled)

6. The implant driver of claim 1, wherein the sterilizable casing is configured to inhibit contamination of external clip whether the external clip is attached to the attachment member or not.

7. The implant driver of claim 1, wherein the external clip is disposed perpendicular to the body.

8. The implant driver of claim 1, further comprising a depth adjustment mechanism for controlling the depth at which the plurality of implants are driven into the surgical site.

9. The implant driver of claim 1, wherein the control element includes a clutch that disengages the driver when a predetermined torque level is reached.

10. The implant driver of claim 1, wherein the control element is further configured to adjust a speed and direction of the driver in response to user input.

11. The implant driver of claim 1, wherein the control element includes a safety interlock mechanism to prevent accidental activation of the driver.

12. The implant driver of claim 1, wherein the plurality of implants are implant screws.

13. (canceled)

14. (canceled)

15. (canceled)

16. The implant driver of claim 2, wherein the distal end receives the attachment member and the driver.

17. (canceled)

18. (canceled)

19. (canceled)

20. The implant driver of claim 16, wherein the sterilizable casing is configured to sterilize the plurality of implants when the external clip is attached to the attachment member.

21. The implant driver of claim 16, wherein the external clip is disposed perpendicular to the body.

22. The implant driver of claim 16, wherein the attachment member includes a grip configured to secure the external clip and magazine during rotational movement of the driver.

23. The implant driver of claim 16, further comprising a depth adjustment mechanism for controlling the depth at which the plurality of implants are driven into the surgical site.

24. The implant driver of claim 16, wherein the control element includes a clutch mechanism that disengages the driver when a predetermined torque level is reached.

25. The implant driver of claim 16, wherein the control element is further configured to adjust a speed and direction of the driver in response to user input.

26. The implant driver of claim 16, wherein the control element includes a safety interlock mechanism to prevent accidental activation of the driver.

27.-42. (canceled)

43. An implant driver, comprising:
a body having a proximal end and a distal end opposite the proximal end, the body including a grip disposed at the proximal end that extends perpendicular to the body;
a barrel assembly mounted to the body;
a circular clip disposed at the distal end of the body, the clip configured to hold a plurality of implants, the clip configured to feed the implant into the barrel for implantation;
a driver operatively connected to the clip, the driver configured to engage and eject the plurality of implants from the clip into a surgical site;
a sterilizable casing configured to enclose the clip; and
a control element configured to activate movement of the driver to drive the implant into the surgical site.

44. (canceled)

45. (canceled)

46. The implant driver of claim 43, wherein the sterilizable casing is configured to sterilize the plurality of implants when the clip is attached to the distal end of the body.

47. The implant driver of claim 43, further comprising a depth adjustment mechanism for controlling the depth at which the plurality of implants are driven into the surgical site.

48. The implant driver of claim 43, wherein the control element includes a safety interlock mechanism to prevent accidental activation of the driver.

49. (canceled)

50. (canceled)

51. (canceled)

52. (canceled)

53. An implant driver, comprising:
a body having a proximal end and a distal end opposite the proximal end, the body including a grip disposed at the proximal end that extends perpendicular to the body;
a barrel assembly mounted to the body;
a plurality of circular clips disposed at the distal end of the body, the plurality of clips configured to hold a plurality of implants, the clips configured to feed the implant into the barrel for implantation;
a driver operatively connected to the plurality of clips, the driver configured to engage and eject the plurality of implants from the plurality of clips into a surgical site;
a sterilizable casing configured to enclose the plurality of clips; and
a control element configured to activate movement of the driver to drive the implant into the surgical site.

54. (canceled)

55. (canceled)

56. The implant driver of claim 53, wherein the sterilizable casing is configured to sterilize the plurality of implants when the clip is attached to the distal end of the body.

57. The implant driver of claim 53, further comprising a depth adjustment mechanism for controlling the depth at which the plurality of implants are driven into the surgical site.

58. The implant driver of claim 53, wherein the control element includes a safety interlock mechanism to prevent accidental activation of the driver.

59.-63. (canceled)
